(19) United States
(12) Patent Application Publication
(10) Pub. No.: US 2021/0262966 A1
(43) Pub. Date: Aug. 26, 2021

(54) ENZYME-BASED DISSOLVED CARBON MONOXIDE SENSOR

(71) Applicant: GWANGJU INSTITUTE OF SCIENCE AND TECHNOLOGY, Gwangju (KR)

(72) Inventors: In Seop CHANG, Gwangju (KR); Simai Reginald Iggan STACY, Gwangju (KR); Yoo Seok LEE, Gwangju (KR); Hye Ryeong LEE, Gwangju (KR); Nu Lee JANG, Gwangju (KR)

(21) Appl. No.: 17/256,193

(22) PCT Filed: Jun. 26, 2019
(86) PCT No.: PCT/KR2019/007767

§ 371 Date: Dec. 26, 2020

(30) Foreign Application Priority Data
Jun. 27, 2018 (KR) 10-2018-00742741

(51) Int. Cl. G01N 27/12; G01N 33/18

(52) U.S. Cl. CPC G01N 27/127; G01N 33/182

(57) ABSTRACT

A dissolved carbon monoxide sensor includes an electrode and a carbon monoxide dehydrogenase fixed on the electrode. According to this configuration, the dissolved carbon monoxide sensor directly detects a dissolved carbon monoxide concentration in a solution by an enzyme reaction of the carbon monoxide dehydrogenase.

### TECHNICAL FIELD

The present invention relates to a carbon monoxide sensor, and more specifically, to an enzyme-based carbon monoxide sensor.

### BACKGROUND ART

Gaseous emissions from a thermoelectric power plant or synthesis gas generated by gasifying biomass and municipal solid waste primarily consists of carbon monoxide (CO), hydrogen (H₂), and carbon dioxide (CO₂) and can be fermented using a biological catalyst so as to produce various types of fuels and create added value. The synthesis gas attracts more attention due to use of microorganisms specially engineered to increase production of high-value chemicals such as organic acids and alcohol from the synthesis gas, and high products selectivity can be provided. In addition, gas carbon is converted into a fuel and a chemical, and thereby an effect of waste disposal on the environment can be reduced.

One of major obstacles in commercialization of synthesis gas fermentation is that gas-liquid substance transfer is limited due to a low dissolved gas concentration, especially, low solubility of CO, in a microorganism cultivated fermentation solution. Activation of microorganisms in a bioreactor changes depending on a dissolved gas concentration. Consequently, an actually dissolved CO concentration is important information for estimation, prediction, and optimization of a substrate consumption rate or product productivity by an operation of a bioreactor.

In the related art, a technology used for measuring a dissolved CO concentration is based on gas chromatography in a synthesis gas fermentation study. The technology is based on a method of indirectly measuring CO decomposed in an aqueous phase, by CO partial pressure of Henry's law and headspace, and it is difficult to measure a real-time dissolved CO concentration. An example of a less common method of directly measuring a CO concentration in an aqueous sample includes myoglobin-protein bioanalysis. However, the method is used offline, is difficult to conduct, and is limitedly used since an error occurs when the method is inaccurately conducted. Consequently, there is a demand for development of a technology for detecting a real-time dissolved CO concentration.

In addition, a low dissolved CO concentration in a synthesis gas fermenting system leads to a limitation on transfer of a substrate to an enzyme electrode. A thickness of an enzyme film on a surface of an enzyme electrode and accessibility of an immobilized enzyme to a substrate are major factors influencing substrate transfer efficiency, and thus there is a demand for development of an enzyme-based dissolved carbon monoxide sensor in which enzymes are fixed on an electrode structure that has a thickness of an enzyme film which is similar to a size of an enzyme molecule and enables the immobilized enzymes to easily come to contact with a substrate aqueous solution.

### CITATION LIST

### Patent Literature

### [Patent Literature 1]

Korean Patent No. 10-1772988

### SUMMARY OF INVENTION

### Technical Problem

A technical object to be achieved by the present invention is to provide a sensor that is capable of directly detecting carbon monoxide dissolved in a liquid.

Another technical object to be achieved by the present invention is to provide a sensor that is capable of detecting carbon monoxide dissolved in a liquid in real time.

Technical objects to be achieved by the present invention are not limited to the technical objects mentioned above, and the following description enables other unmentioned technical objects to be clearly understood by a person of ordinary skill in the art to which the present invention belongs.

### Solution to Problem

In order to achieve the technical object, an embodiment of the present invention provides a dissolved carbon monoxide sensor.

According to the embodiment, the dissolved carbon monoxide sensor may include a nanopatterned electrode and a carbon monoxide dehydrogenase fixed on the nanopatterned electrode.

According to the embodiment, the dissolved carbon monoxide sensor may directly detect a dissolved carbon monoxide concentration in a solution by an enzyme reaction of the carbon monoxide dehydrogenase.

According to the embodiment, the carbon monoxide dehydrogenase may directly transfer electrons generated by the enzyme reaction to the electrode.

According to the embodiment, the electrode may contain Pt, Cu, Zn, Fe, Ni, Co, Mn, Au, Ag, carbon fiber, carbon nanotube, graphene, or graphite.

According to the embodiment, the nanopatterned electrode may have a sub-wavelength nanostructure obtained using a self-masked dry etching technique.

According to the embodiment, the nanopatterned electrode may have a pyramid-shaped pattern.

According to the embodiment, the pyramid-shaped pattern may have a height of 10 nm to 200 nm.

According to the embodiment, the pyramid-shaped pattern may have intervals of 10 nm to 200 nm.

According to the embodiment, the carbon monoxide dehydrogenase may contain an L unit at which an active site is positioned, an M unit coupled to the L unit, and an S unit coupled to the M unit.

According to the embodiment, the carbon monoxide dehydrogenase may be fixed on the nanopatterned electrode by a metal-immobilized peptide expressed at the L unit, the M unit, or the S unit.

According to the embodiment, the carbon monoxide dehydrogenase may be fixed on the electrode by a printing method, a dipping method, or an immersing method.

In the dissolved carbon monoxide sensor, the enzyme reaction may cause a reaction represented by the following chemical formula (1).

CO+H₂O→CO₂+2H⁺+2e⁻ Chemical Formula (1)

In order to achieve the other technical objects, another embodiment of the present invention provides a method for detecting dissolved carbon monoxide.

According to the other embodiment, the method for detecting dissolved carbon monoxide may include: a step of electrically connecting the dissolved carbon monoxide sensor according to the embodiment of the present invention to a current value detector; a step of immersing the dissolved carbon monoxide sensor connected to the detector into an analysis target liquid; a step of applying voltage to the dissolved carbon monoxide sensor immersed into the liquid; and a step of detecting a current change by the detector, the current change occurring due to the enzyme reaction of the dissolved carbon monoxide sensor.

In the method for detecting dissolved carbon monoxide, carbon monoxide dissolved in the analysis target liquid may be detected in real time.

In the method for detecting dissolved carbon monoxide, the enzyme reaction may cause a reaction represented by the following chemical formula (1).

CO+H₂O→CO₂+2H⁺+2e⁻ Chemical Formula (1)

According to the other embodiment, the analysis target liquid may have pH of 6.5 to 7.5.

### Advantageous Effects of Invention

According to an embodiment of the present invention, it is possible to provide a sensor that is capable of directly detecting carbon monoxide dissolved in a liquid.

According to the embodiment of the present invention, it is possible to provide a sensor that is capable of detecting carbon monoxide dissolved in a liquid in real time.

According to the embodiment of the present invention, it is possible to provide a dissolved carbon monoxide sensor that is capable of measuring a concentration in a wide range by a reduction in resistance due to a limitation on substrate transfer.

According to the embodiment of the present invention, it is possible to provide a dissolved carbon monoxide sensor that performs highly accurate detection.

The effects of the present invention are construed not to be limited to the above-mentioned effects but to include every effect that can be derived from configurations of the invention described in the detailed description of the embodiments or claims of the present invention.

### BRIEF DESCRIPTION OF DRAWINGS

FIG. 1 a view illustrating a dissolved carbon monoxide sensor according to an embodiment of the present invention.

FIG. 2 is a view illustrating a dissolved carbon monoxide sensor according to another embodiment of the present invention.

FIG. 3 is a view illustrating a dissolved carbon monoxide sensor according to still another embodiment of the present invention.

FIG. 4 is a schematic view illustrating a process of manufacturing a nanopatterned electrode having a sub-wavelength nanostructure obtained using a self-masked dry etching technique.

FIG. 5 illustrates a cyclic voltammogram of the dissolved carbon monoxide sensor according to the embodiment of the present invention.

FIG. 6 illustrates scan rate-current graphs of the dissolved carbon monoxide sensor according to the embodiment of the present invention.

FIG. 7 illustrates a cyclic voltammogram and a scan rate-current graph of the carbon monoxide sensor according to the embodiment of the present invention.

FIG. 8 is a flowchart illustrating a method for detecting dissolved carbon monoxide according to another embodiment of the present invention.

FIG. 9 illustrates an enzyme loading amount-current graph obtained from detection performed by the method for detecting dissolved carbon monoxide according to the other embodiment of the present invention.

FIG. 10 is a CO partial pressure-current graph obtained from detection performed by the method for detecting dissolved carbon monoxide according to the other embodiment of the present invention.

FIG. 11 illustrates a dissolved CO concentration-current graph obtained from detection performed by the method for detecting dissolved carbon monoxide according to the other embodiment of the present invention.

### DESCRIPTION OF EMBODIMENTS

Hereinafter, the present invention will be described with reference to the accompanying drawings. However, the present invention can be realized as various different embodiments, thus not being limited to embodiments described here. Besides, a part irrelevant to the description is omitted from the drawings in order to clearly describe the present invention, and similar reference signs are assigned to similar parts through the entire specification.

In the entire specification, a case where a certain part “is coupled to (accesses, is in contact with, or is connected to)” another part includes not only a case where the parts are “directly coupled” to each other, but also a case where the parts are “indirectly coupled” to each other with another member interposed therebetween. In addition, a case where a certain part “includes” a certain configurational element means that another configurational element is not excluded but can be further included, unless specifically described otherwise.

Terms used in this specification are only used to describe a specific embodiment and are not intentionally used to limit the present invention thereto. A word as a singular noun also includes a meaning of its plural noun, unless obviously implied otherwise in context. In this specification, words such as “to include” or “to have” are construed to specify that a feature, a number, a step, an operation, a configurational element, a member, or a combination thereof described in the specification is present and not to exclude presence or a possibility of addition of one or more additional features, numbers, steps, operations, configurational elements, members, or combinations thereof in advance.

Hereinafter, embodiments of the present invention will be described in detail with reference to the accompanying drawings.

A dissolved carbon monoxide sensor according to an embodiment of the invention is described.

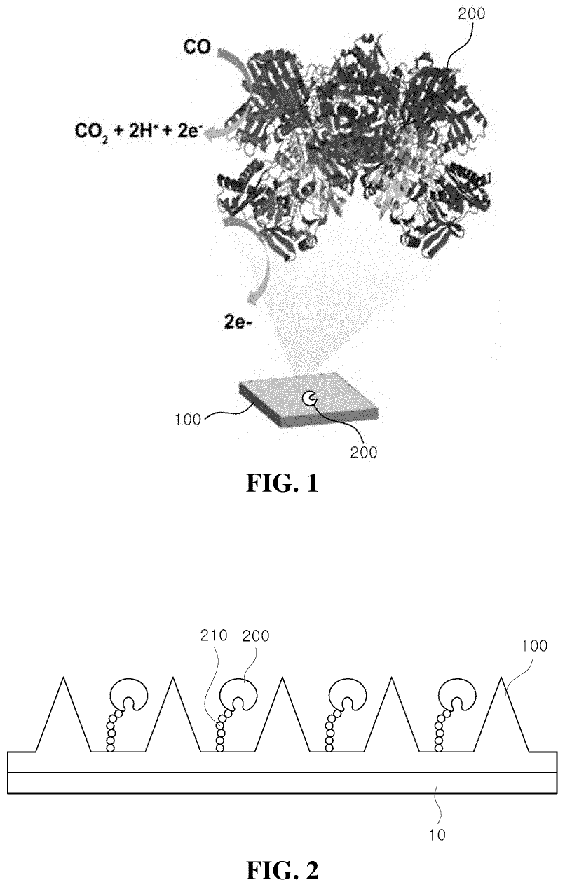

FIG. 1 a view illustrating the dissolved carbon monoxide sensor according to an embodiment of the present invention.

With reference to FIG. 1, the dissolved carbon monoxide sensor can include a nanopatterned electrode 100 and a carbon monoxide dehydrogenase 200 fixed on the nanopatterned electrode 100.

Here, the dissolved carbon monoxide sensor directly detects a dissolved carbon monoxide concentration in a solution by an enzyme reaction of the carbon monoxide dehydrogenase 200.

Here, the carbon monoxide dehydrogenase 200 directly transfers electrons generated by the enzyme reaction to the electrode.

Here, the electrode 100 can contain Pt, Cu, Zn, Fe, Ni, Co, Mn, Au, Ag, carbon fiber, carbon nanotube, graphene, or graphite.

Here, the nanopatterned electrode has a sub-wavelength nanostructure obtained using a self-masked dry etching technique.

Here, the sub-wavelength nanostructure means a wavelength shaped structure in which both a main wave and a sub-wave are present.

Here, the nanopatterned electrode has a pyramid-shaped pattern.

Here, the nanopatterned electrode has the sub-wavelength nanostructure obtained using a self-masked dry etching technique and has the pyramid-shaped pattern, and thereby carbon monoxide dehydrogenases are uniformly settled between the patterns to prevent enzymes from being clumped together such that substrate transferability can improve.

Here, it is preferable that a height and intervals of the nanopatterns be approximate to a size of the carbon dioxide dehydrogenase. When the height and the intervals of the nanopatterns are approximate to the size of the carbon dioxide dehydrogenase, the carbon monoxide dehydrogenases can be uniformly applied between the nanopatterns, and thus smooth substrate transfer can be induced.

Preferably, the pyramid-shaped patterns have a height of 10 nm to 200 nm.

Here, when the pyramid-shaped patterns have a height of smaller than 10 nm, the carbon monoxide dehydrogenases are not uniformly applied on the nanopatterned electrodes, and clumping of the enzymes can occur.

Here, when the pyramid-shaped patterns have a height of greater than 200 nm, the carbon monoxide dehydrogenases are not uniformly applied on the nanopatterned electrodes, and clumping of the enzymes can occur.

Preferably, the pyramid-shaped patterns have intervals of 10 nm to 200 nm.

Here, when the pyramid-shaped patterns have intervals of smaller than 10 nm, the carbon monoxide dehydrogenases are not uniformly applied on the nanopatterned electrodes, and clumping of the enzymes can occur.

Here, when the pyramid-shaped patterns have intervals of greater than 200 nm, the carbon monoxide dehydrogenases are not uniformly applied on the nanopatterned electrodes, and clumping of the enzymes can occur.

In the embodiment of the present invention, the pyramid-shaped patterns enable the carbon monoxide dehydrogenases to be uniformly applied. In general, an enzyme has a diameter of 50 nm to 200 nm, and in order for the carbon monoxide dehydrogenases to be uniformly applied on the pyramid-shaped patterns, it is preferable that the pyramid-shaped pattern have the height and the intervals approximate to the size of the enzyme. When the pattern has the height and the intervals approximate to the size of the enzyme, the enzymes are settled between the patterns such that the enzymes can be uniformly applied on the patterned electrode. Consequently, when the pattern has the height and the intervals much smaller or greater than a size range of the enzymes, it is difficult for the enzymes to be separately settled between the patterns, and thus a phenomenon in which the enzymes clump together can occur.

FIG. 2 is a view illustrating a dissolved carbon monoxide sensor according to another embodiment of the present invention.

With reference to FIG. 2, the dissolved carbon monoxide sensor according to the embodiment of the present invention can include a substrate 10, an electrode 100 positioned on the substrate 10, and a carbon monoxide dehydrogenase 200 fixed on the electrode 100.

Here, the carbon monoxide dehydrogenase 200 is fixed on the electrode 100 by a metal-immobilized peptide 210 expressed at the carbon monoxide dehydrogenase 200.

Here, the electrode 100 can be a pattern-formed electrode.

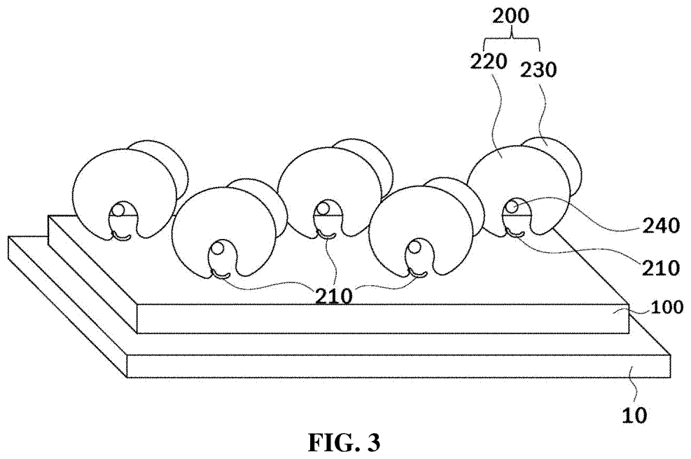

FIG. 3 is a view illustrating a dissolved carbon monoxide sensor according to still another embodiment of the present invention.

With reference to FIG. 3, the dissolved carbon monoxide sensor according to the embodiment of the present invention can include a substrate 10, an electrode 100 positioned on the substrate 10, and a carbon monoxide dehydrogenase 200 fixed on the electrode 100.

Here, the carbon monoxide dehydrogenase 200 is fixed on the electrode 100 by a metal-immobilized peptide 210 expressed at the carbon monoxide dehydrogenase 200.

Here, the carbon monoxide dehydrogenase 200 contains an L unit 220 at which an active site is positioned and an M unit 230 coupled to the L unit 220.

Here, the metal-immobilized peptide 210 is formed at one of the L unit 220 or the M unit 230.

Here, the carbon monoxide dehydrogenase 200 can contain a cofactor 240 at the L unit 220 at which the active site is positioned.

Here, the cofactor 240 can be added to promote an enzyme reaction of the carbon monoxide dehydrogenase 200.

The dissolved carbon monoxide sensor according to the embodiment of the present invention detects dissolved carbon monoxide depending on a current change due to electrons generated by a chemical reaction occurring at the active site of the enzyme. Here, in order to improve performance of the dissolved carbon monoxide sensor, it is important to effectively transfer the electrons generated at the active site of the enzyme to the electrode. Here, in order to effectively transfer the electrons generated at the active site of the enzyme to the electrode, it is important to shorten a distance between the active site of the enzyme and the electrode.

In the dissolved carbon monoxide sensor according to the embodiment of the present invention, the metal-immobilized peptide is expressed at an L sub-unit at which the active site of the enzyme is positioned, an M sub-unit, or an S sub-unit to be directly fixed on the electrode, and thereby a distance between the active site and the electrode is closely fixed.

A method in which an enzyme transfers an electron to an electrode can be divided into a mediated electron transfer (MET) method and a direct electron transfer (DET) method, and a problem arises in the MET method in that an electron potential is lowered due to an intermediate medium. The problem arises because an electron transfer distance is very important for efficient electron transfer, but the electron transfer distance increases due to the intermediate medium in the MET method. In the present invention, since the metal-immobilized peptide expressed at the carbon monoxide dehydrogenase is directly fixed to a metal electrode pattern, the carbon monoxide dehydrogenase can be very closely fixed to the metal electrode pattern. Hence, the DET method can be conducted, and thus a high electron potential can be maintained.

The electron transfer efficiency depending on the electron transfer distance can be determined by the following expression (1).

$${K}_{et} = {10}^{13} {e}^{- 0.91 ( d \text{-} 3 )} {e}^{[ \frac{- ( Δ G + λ )}{4 RT λ} ]} \qquad Expression ( 1 )$$

(In Expression (1), Kₑₜ represents an electron transfer rate constant, d represents an actual electron transfer distance, G represents free energy, X represents reconstruction energy.)

Consequently, in the dissolved carbon monoxide sensor according to the embodiment of the present invention, the carbon monoxide dehydrogenase is directly fixed to the electrode using the metal-immobilized peptide expressed at the carbon monoxide dehydrogenase, and thereby the distance between the active site of the enzyme and the electrode is shortened such that the performance of the dissolved carbon monoxide sensor can improve.

Consequently, in the dissolved carbon monoxide sensor according to the embodiment of the present invention, the active site of the enzyme and the electrode are fixed to be close to each other by the metal-immobilized peptide, and thereby the electron transfer efficiency can improve.

Here, the carbon monoxide dehydrogenase 200 is fixed on the electrode by a printing method, a dipping method, or an immersing method.

Here, in the dissolved carbon monoxide sensor, the enzyme reaction causes a reaction represented by the following chemical formula (1).

CO+H₂O→CO₂+2H⁺+2e⁻ Chemical Formula (1)

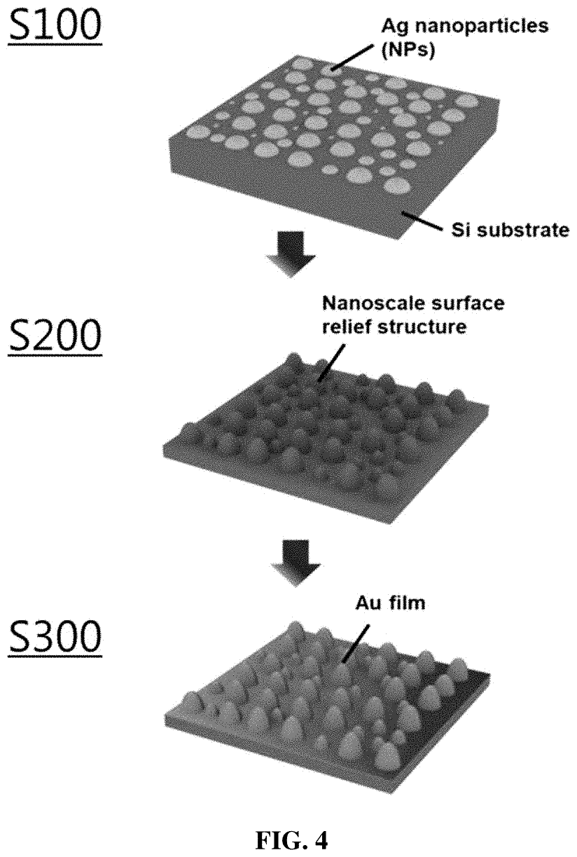

FIG. 4 is a schematic view illustrating a process of manufacturing a nanopatterned electrode having the sub-wavelength nanostructure obtained using a self-masked dry etching technique.

With reference to FIG. 4, first, silver nanoparticles (Ag nanoparticles) formed a pattern on a silicon substrate (Si substrate) (S100).

Next, the silicon substrate was etched by dry etching to form a silicon substrate on which a sub-wavelength nanostructure pattern was formed (S200).

Next, gold (Au) was deposited on the silicon substrate on which the sub-wavelength nanostructure pattern was formed such that the nanopatterned electrode having the sub-wavelength nanostructure was formed (S300).

### Embodiment 1

Gold-patterned electrodes having a size of 1 cm² were agitated in 3 ml of 50 mM PB buffer containing 200 μl of CODH enzymes and were immersed therein for one hour, and the dissolved carbon monoxide sensor according to the embodiment of the present invention was manufactured.

### Experimental Example 1

First, deionized water was subjected to bubbling with CO for 30 min. at room temperature such that a CO-saturated standard solution was produced, and a CO content was calculated to be 0.95 mM by saturated solubility.

Cyclic voltammetry was measured by a potentiometer using the dissolved carbon monoxide sensor manufactured by Embodiment 1, a platinum (Pt) wire, and a three-electrode system made of Ag/AgCl.

Here, a partial amount of the CO-saturated standard solution was continuously added.

Here, the cyclic voltammetry was conducted by a gas-tight electrochemical cell in conditions of 30° C. and 50 mM PB (pH 7.2).

Regarding current measurement, after a steady-state current was reached, data was recorded in real time.

### Experimental Example 2

First, deionized water was subjected to bubbling with CO for 30 min. at room temperature such that a CO-saturated standard solution was produced, and a CO content was calculated to be 0.95 mM by saturated solubility.

Cyclic voltammetry was measured by a potentiometer using a gold electrode, the platinum (Pt) wire, and the three-electrode system made of Ag/AgCl.

Here, a partial amount of the CO-saturated standard solution was continuously added.

Here, the cyclic voltammetry was conducted by a gas-tight electrochemical cell in conditions of 30° C. and 50 mM PB (pH 7.2).

Regarding current measurement, after a steady-state current was reached, data was recorded in real time.

### Experimental Example 3

Cyclic voltammetry was measured in the same manner as in Experimental Example 1 except that a partial amount of the CO-saturated standard solution in Experimental Example 1 was not added.

### Experimental Example 4

Cyclic voltammetry was measured in the same manner as in Experimental Example 1 except that a partial amount of the CO-saturated standard solution in Experimental Example 2 was not added.

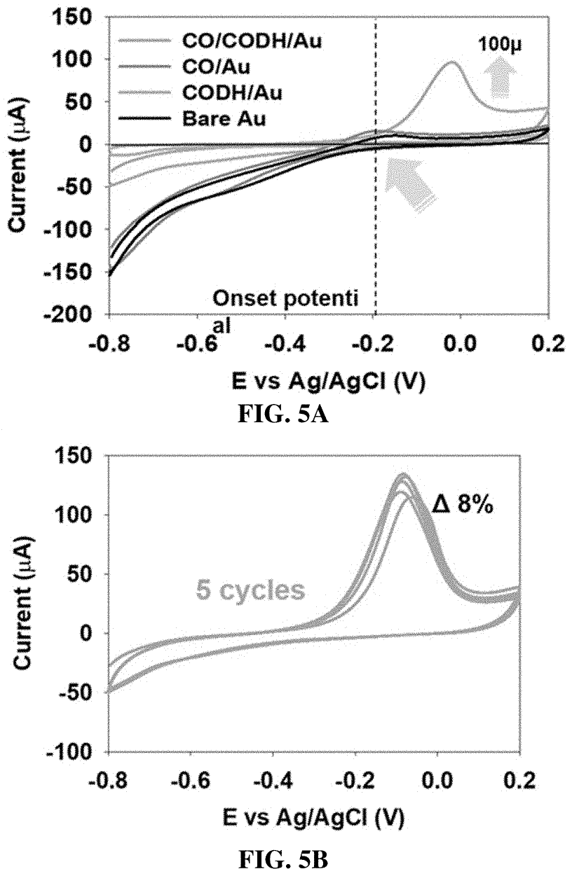

Results of Experimental Examples 1 to 4 are shown on graphs in FIG. 5.

FIG. 5 illustrates a cyclic voltammogram of the dissolved carbon monoxide sensor according to the embodiment of the present invention.

FIG. 5(a) is a cyclic voltammogram of Experimental Examples 1 to 5. Here, CO/CODH/Au represents a result value of Experimental Example 1, CO/Au represents a result value of Experimental Example 2, CODH/Au represents a result value of Experimental Example 3, and Bare Au represents a result value of Experimental Example 4. FIG. 5(a) clarifies that when CO is not present, or when the dissolved carbon monoxide sensor having the enzyme according to the embodiment of the present invention is not provided, an oxidation-reduction peak does not appear in a potential range. In addition, in Experimental Example 1 according to the embodiment of the present invention, an oxidation-reduction peak of 100 μA or higher is found to be present.

FIG. 5(b) is a graph illustrating repeated measurement of an experiment of Experimental Example 1 during five cycles at a scan rate of 50 mVs⁻¹. FIG. 5(b) clarifies that when the sensor according to the embodiment of the present invention is used, an anode peak current having a relative standard deviation of lower than 8% is checked even when repeated measurement is conducted, and thus stability of the dissolved carbon monoxide sensor according to the embodiment of the present invention is confirmed.

### Experimental Example 5

In order to check the effects depending on the current scan rate, the experiment of Experimental Example 1 was conducted by changing the scan rate in a range from 10 mVs⁻¹ to 100 mVs⁻¹.

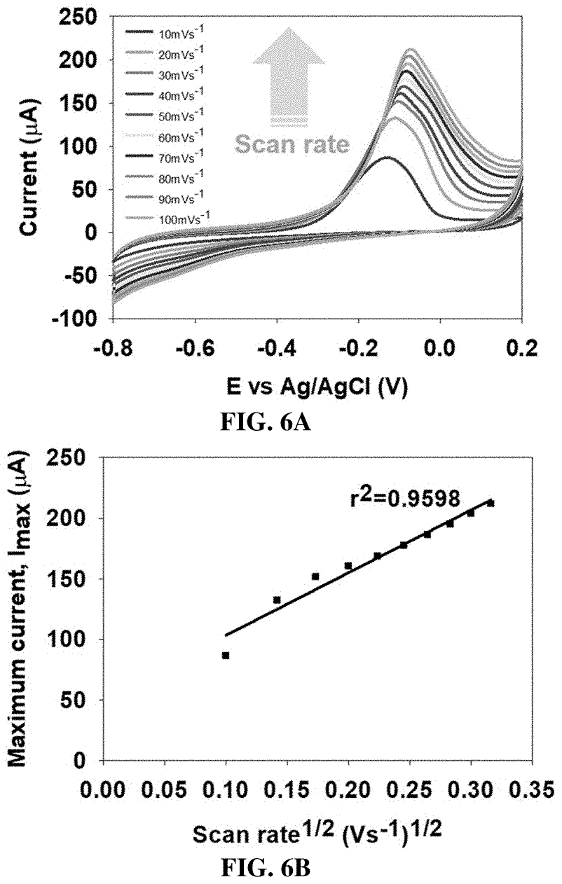

Results thereof are shown on graphs in FIG. 6.

FIG. 6 illustrates scan rate-current graphs of the dissolved carbon monoxide sensor according to the embodiment of the present invention.

FIG. 6(a) is a cyclic voltammogram at various scan rates. FIG. 6(b) is a graph obtained by plotting anode current peaks with respect to the maximum current value depending on the scan rates.

FIG. 6 clarifies that the oxidation-reduction current peak, the maximum current value, and the scan rate have a linear relationship. This indicates that an electron direct-transfer system between enzymes and electrodes of the sensor according to the embodiment of the present invention depends on a surface control process.

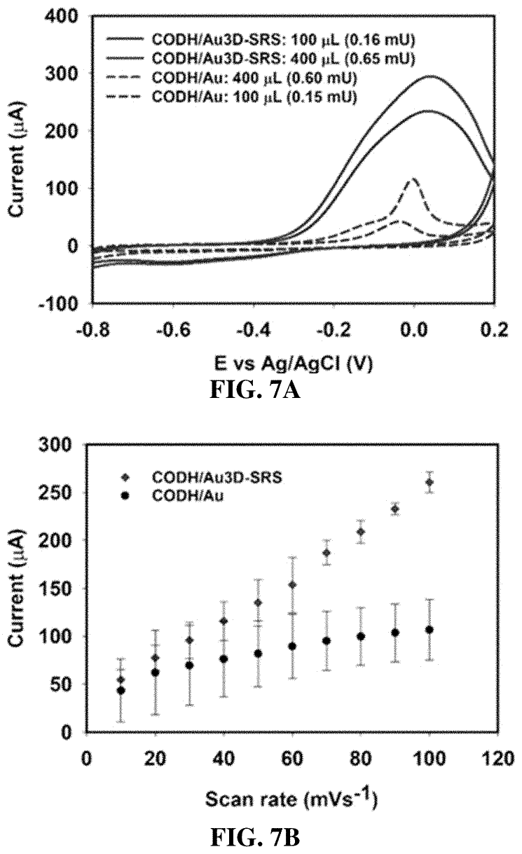

FIG. 7 illustrates a cyclic voltammogram and a scan rate-current graph of the carbon monoxide sensor according to the embodiment of the present invention.

The dissolved carbon monoxide sensor according to the embodiment of the present invention is an enzyme-based biosensor and detects dissolved carbon monoxide by measuring a current value that changes depending on a dissolved carbon monoxide concentration.

The enzyme-based biosensor attracts much attention with high sensitivity and high selectivity due to enzyme substrate specificity, the miniaturization, and the mass production possibility. Consequently, the dissolved carbon monoxide sensor according to the embodiment of the present invention can provide a sensor that has high selectivity for carbon monoxide using the enzyme which generates electrons with carbon monoxide as a substrate.

A detection principle of the enzyme-based biosensor for measuring a current value is based on electron transfer (ET) between the active site of the enzyme and an electrode surface having an action potential. An electron transfer method of the enzyme-based sensor includes the direct electron transfer (DET) method and the mediated electron transfer (MET) method. Of the methods, the direct electron transfer method has a simpler electron moving path and a faster response speed than those of the mediated electron transfer method.

Electron transfer efficiency between the enzyme active site and the electrode surface significantly influences performance of a bioelectrochemical apparatus, an enzyme fuel cell, a biosensor, and a photosynthesis apparatus. Consequently, in order to provide an enzyme-based sensor having high electron transfer efficiency, it is desirable to use enzymes which can directly transfer electrons.

Consequently, according to the embodiment of the present invention, it is possible to provide a sensor that is capable of directly detecting carbon monoxide dissolved in a liquid by an enzyme reaction.

In addition, the dissolved carbon monoxide sensor according to the embodiment of the present invention can provide a dissolved carbon monoxide sensor that has a fast response speed using the enzymes which can directly transfer electrons to the electrode.

Further, the dissolved carbon monoxide sensor according to the embodiment of the present invention is capable of monitoring a concentration of carbon monoxide (CO) dissolved in a liquid in real time.

A method for detecting dissolved carbon monoxide according to another embodiment of the present invention is described.

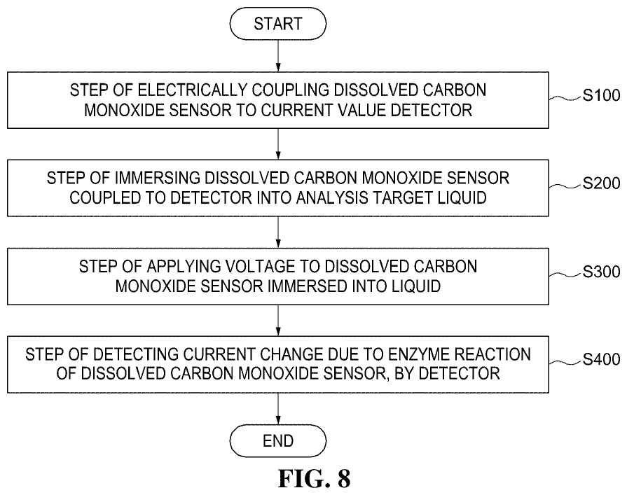

FIG. 8 is a flowchart illustrating the method for detecting dissolved carbon monoxide according to the other embodiment of the present invention.

With reference to FIG. 8, the method for detecting dissolved carbon monoxide can include Step S100 of electrically connecting the dissolved carbon monoxide sensor to a current value detector according to the embodiment of the present invention, Step S200 of immersing the dissolved carbon monoxide sensor connected to the detector into an analysis target liquid, Step S300 of applying voltage to the dissolved carbon monoxide sensor immersed into the liquid, and Step S400 of detecting a current change by the detector, the current change occurring due to the enzyme reaction of the dissolved carbon monoxide sensor.

Here, in the method for detecting dissolved carbon monoxide, carbon monoxide dissolved in the analysis target liquid is detected in real time.

Here, in the method for detecting dissolved carbon monoxide, the enzyme reaction causes a reaction represented by the following chemical formula (1).

CO+H₂O→CO₂+2H⁺+2e⁻ Chemical Formula (1)

Here, the analysis target liquid has pH of 6.5 to 7.5.

### Experimental Example 6

An effect of an amount of enzymes contained in the sensor during detection of dissolved carbon monoxide was evaluated.

First, the dissolved carbon monoxide sensor was manufactured by loading 100 μl, 200 μl, and 400 μl of CODH on respective electrode surfaces. Here, enzymes were fixed on gold (Au) electrodes at concentrations of 0.147 mU, 0.293 mU, and 0.586 mU, respectively.

Next, CV measurement was conducted at potentials of −0.8 V to +0.2 V (pH 7.2). Here, PB and CO contents of an electrochemical cell were calculated to be 0.95 mM by the saturated solubility.

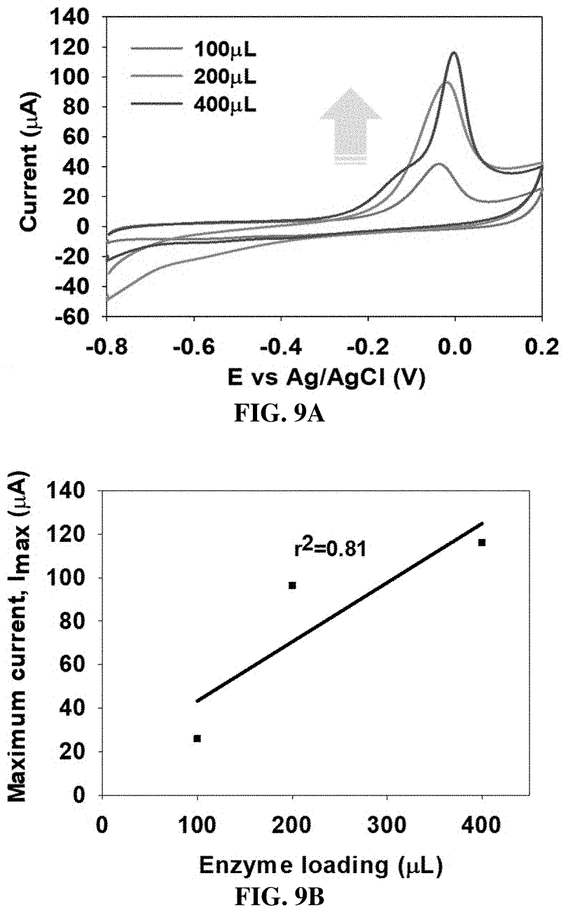

Results thereof are shown on graphs in FIG. 9.

FIG. 9 illustrates an enzyme loading amount-current graph obtained from detection performed by the method for detecting dissolved carbon monoxide according to the other embodiment of the present invention.

FIG. 9(a) is a cyclic voltammogram according to enzyme loading amounts, and FIG. 9(b) is a graph illustrating maximum current values depending on amounts of enzymes.

FIG. 9 clarifies that the oxidation-reduction current peaks increase according to the amount of enzymes. This indicates that the oxidation-reduction reaction depends on the amount of enzymes. In addition, the following can be checked. The maximum current value significantly increases as the loading amount increases from 100 μl to 200 μl, whereas the maximum current value decreases when the loading amount increases from 200 μl to 400 μl. This indicates that the enzyme loading of greater than 400 μl does not bring about a significant result.

### Experimental Example 7

Analytical performance of a detection method using the dissolved carbon monoxide sensor according to the other embodiment of the present invention was evaluated.

For evaluation, cyclic voltammetry (CV) was measured at carbon monoxide (CO) partial pressure of 0.5 psi, 10 psi, and 15 psi.

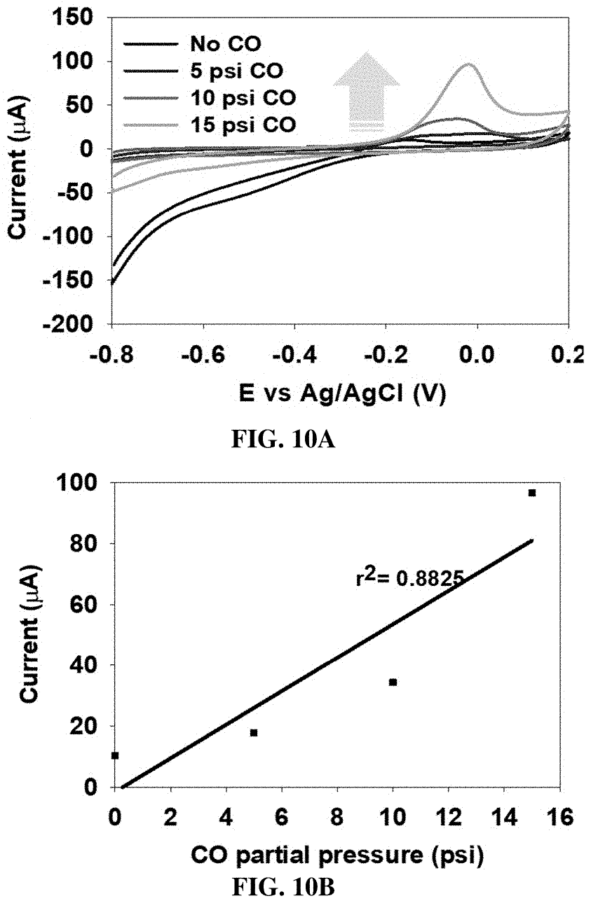

According to Henry's law, a concentration of solute gas in a solution is directly proportionate to partial pressure of gas over the solution. Consequently, as the CO partial pressure increases, a concentration of dissolved CO increases. Consequently, the analytical performance of the biosensor with respect to the dissolved carbon monoxide was evaluated by conducting the CV at different carbon monoxide partial pressures. Results thereof are shown on graphs in FIG. 10.

FIG. 10 is a CO partial pressure-current graph obtained from detection performed by the method for detecting dissolved carbon monoxide according to the other embodiment of the present invention.

FIG. 10(a) is a graph illustrating a cyclic voltammogram at various CO partial pressures, and FIG. 10(b) is a graph illustrating maximum currents depending on the CO partial pressures. FIG. 10 clarifies that similar CV patterns are observed even when the CO partial pressures are different, but peak oxidation currents have a linear relationship with the CO partial pressures in a specific potential range. This indicates that a level of oxidation-reduction reaction of the enzyme is proportionate to a concentration of dissolved carbon monoxide.

### Experimental Example 8

Current measuring performance of the method for detecting dissolved carbon monoxide according to the other embodiment of the present invention with respect to the carbon monoxide concentration was tested.

Here, the current was measured in the same method as in Experimental Example 1, and voltage of −0.02 V was applied at a scan rate of 50 mVs⁻¹.

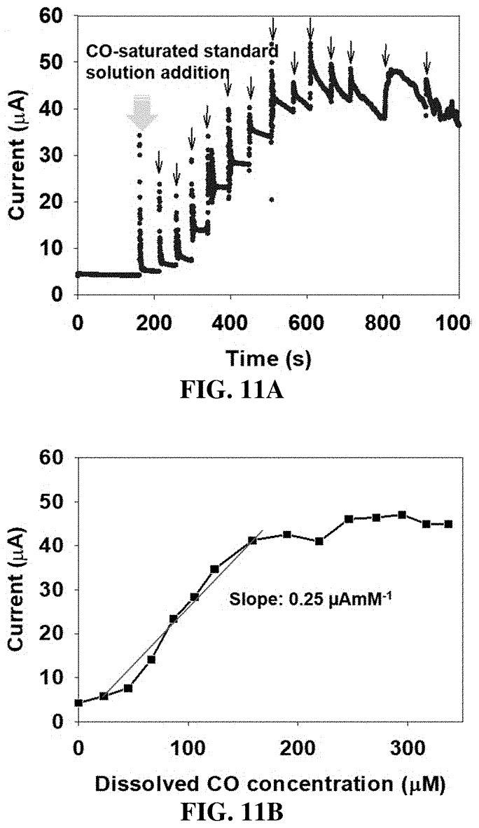

Here, the carbon monoxide concentration was adjusted by sequential addition of 23 μM to 335 μM of CO-saturated standard solution (PB), and a signal indicating a normal state was generated within five sec. in each case. Results thereof are shown on graphs in FIG. 11.

FIG. 11 illustrates a dissolved CO concentration-current graph obtained from detection performed by the method for detecting dissolved carbon monoxide according to the other embodiment of the present invention.

FIG. 11(a) is a continuous current-current response graph of the dissolved carbon monoxide sensor illustrating spikes (represented by ↓) of the CO-saturated standard solution, and FIG. 11(b) is a graph of current values depending on the dissolved CO concentrations. FIG. 11 clarifies that the current values and the carbon monoxide concentrations are linearly proportionate to each other within a range of dissolved carbon monoxide concentration from 23 μM to 190 μM. At that point, a correlation coefficient is 0.937 which means that reliability is high, and a slop value indicating sensitivity of the sensor is confirmed to be 250 μAmM⁻¹cm⁻². This clarifies that a high response speed and reliability are achieved by the method for detecting dissolved carbon monoxide using the dissolved carbon monoxide sensor according to the other embodiment of the present invention.

In the method for detecting dissolved carbon monoxide according to the other embodiment of the present invention, dissolved carbon monoxide is detected by measuring a current value that changes depending on a dissolved carbon monoxide concentration by using the enzyme-based biosensor.

The enzyme-based biosensor attracts much attention with high sensitivity and high selectivity due to the enzyme substrate specificity, the miniaturization, and the mass production possibility. Consequently, the dissolved carbon monoxide sensor according to the embodiment of the present invention can provide a sensor that has high selectivity for carbon monoxide using the enzyme which generates electrons with carbon monoxide as a substrate.

A detection principle of the enzyme-based biosensor for measuring a current value is based on electron transfer (ET) between the active site of the enzyme and an electrode surface having an action potential. The electron transfer method of the enzyme-based sensor includes the direct electron transfer (DET) method and the mediated electron transfer (MET) method. Of the methods, the direct electron transfer method has a simpler electron moving path and a faster response speed than those of the mediated electron transfer method.

Electron transfer efficiency between the enzyme active site and the electrode surface significantly influences performance of a bioelectrochemical apparatus, an enzyme fuel cell, a biosensor, and a photosynthesis apparatus. Consequently, in order to provide an enzyme-based sensor having high electron transfer efficiency, it is desirable to use enzymes which can directly transfer electrons.

Consequently, according to the embodiment of the present invention, it is possible to detect carbon monoxide dissolved in a liquid by an enzyme reaction.

In addition, in the method for detecting dissolved carbon monoxide according to the other embodiment of the present invention, the fast response speed can be achieved using the enzymes which can directly transfer electrons to the electrode.

Further, in the method for detecting dissolved carbon monoxide according to the other embodiment of the present invention, a concentration of carbon monoxide (CO) dissolved in a liquid can be monitored in real time.

The description of the present invention described above is provided as an example, and a person of ordinary skill in the art to which the present invention belongs can understand that it is possible to easily modify the present invention to another embodiment without changing the technical idea or an essential feature of the present invention. Therefore, the embodiments described above need to be understood as exemplified embodiments in every aspect and not as embodiments to limit the present invention. For example, configurational elements described in a single form can be realized in a distributed manner. Similarly, the configurational elements described in the distributed manner can be realized in a combined manner.

The scope of the present invention needs to be represented by the claims to be described below, and meaning and the scope of the claims and every modification or modified embodiment derived from an equivalent concept of the claims need to be construed to be included in the scope of the present invention.

### REFERENCE SIGNS LIST

10 SUBSTRATE
100 ELECTRODE
200 CARBON MONOXIDE DEHYDROGENASE
210 METAL-IMMOBILIZED PEPTIDE
220 L UNIT
230 M UNIT
240 COFACTOR

1. A dissolved carbon monoxide sensor comprising:
a nanopatterned electrode; and
a carbon monoxide dehydrogenase fixed on the nanopatterned electrode,
wherein a dissolved carbon monoxide concentration in a solution is directly detected by an enzyme reaction of the carbon monoxide dehydrogenase.

2. The dissolved carbon monoxide sensor according to claim 1,
wherein the carbon monoxide dehydrogenase directly transfers electrons generated by the enzyme reaction to the electrode.

3. The dissolved carbon monoxide sensor according to claim 1,
wherein the nanopatterned electrode contains Pt, Cu, Zn, Fe, Ni, Co, Mn, Au, Ag, carbon fiber, carbon nanotube, graphene, or graphite.

4. The dissolved carbon monoxide sensor according to claim 1,
wherein the nanopatterned electrode has a sub-wavelength nanostructure obtained using a self-masked dry etching technique.

5. The dissolved carbon monoxide sensor according to claim 1,
wherein the nanopatterned electrode has a pyramid-shaped pattern.

6. The dissolved carbon monoxide sensor according to claim 5,
wherein the pyramid-shaped pattern has a height of 10 nm to 200 nm.

7. The dissolved carbon monoxide sensor according to claim 5,
wherein the pyramid-shaped pattern has intervals of 10 nm to 200 nm.

8. The dissolved carbon monoxide sensor according to claim 1,
wherein the carbon monoxide dehydrogenase contains an L unit at which an active site is positioned, an M unit coupled to the L unit, and an S unit coupled to the M unit, and
wherein the carbon monoxide dehydrogenase is fixed on the nanopatterned electrode by a metal-immobilized peptide expressed at the L unit, the M unit, or the S unit.

9. The dissolved carbon monoxide sensor according to claim 1,
wherein the carbon monoxide dehydrogenase is fixed on the nanopatterned electrode by a printing method, a dipping method, or an immersing method.

10. The dissolved carbon monoxide sensor according to claim 1,
wherein the enzyme reaction causes a reaction represented by the following chemical formula (1).
CO+H₂O→CO₂+2H⁺+2e⁻ Chemical Formula (1)

11. A method for detecting dissolved carbon monoxide, comprising:
a step of electrically connecting the dissolved carbon monoxide sensor according to claim 1 to a current value detector;
a step of immersing the dissolved carbon monoxide sensor connected to the detector into an analysis target liquid;
a step of applying voltage to the dissolved carbon monoxide sensor immersed into the liquid; and
a step of detecting a current change by the detector, the current change occurring due to the enzyme reaction of the dissolved carbon monoxide sensor,
wherein carbon monoxide dissolved in the analysis target liquid is detected in real time.

12. The method for detecting dissolved carbon monoxide according to claim 11,
wherein the enzyme reaction causes a reaction represented by the following chemical formula (1).
CO+H₂O→CO₂+2H⁺+⁻ Chemical Formula (1)

13. The method for detecting dissolved carbon monoxide according to claim 11,
wherein the analysis target liquid has pH of 6.5 to 7.5.
